(19) United States
(12) Patent Application Publication
(10) Pub. No.: US 2023/0405275 A1
(43) Pub. Date: Dec. 21, 2023

(54) DEVICES, SYSTEMS, AND METHODS FOR STEERING A CATHETER

(71) Applicants: BOSTON SCIENTIFIC SCIMED, INC., MAPLE GROVE, MN (US); Mayo Foundation for Medical Education and Research, Rochester, MN (US)

(72) Inventors: Mitchell Nelson, Hudson, WI (US); Aaron Abbott, Columbia Heights, MN (US); Joel T. Eggert, Plymouth, MN (US)

(73) Assignees: BOSTON SCIENTIFIC SCIMED, INC., MAPLE GROVE, MN (US); Mayo Foundation for Medical Education and Research, Rochester, MN (US)

(21) Appl. No.: 18/211,549

(22) Filed: Jun. 19, 2023

Related U.S. Application Data
(60) Provisional application No. 63/354,015, filed on Jun. 21, 2022.

(51) Int. Cl. A61M 25/01

(52) U.S. Cl. CPC A61M 25/0136; A61M 25/0147

(57) ABSTRACT

A steering system for a steerable flexible elongate member such as for delivering a medical device and/or system. The steering system may be configured for two-way or four-way steering. A control knob is rotatable to control steering of the steerable flexible elongate member in a first direction or a second direction within a steering plane. In a four-way steering system, a first control knob steers the steerable flexible elongate member in a first steering plane, and a second control knob steers the steerable flexible elongate member in a second steering plane transverse to the first steering plane. The steering system may be supported on a stand configured to allow rotational support of the steering system. The stand may support two steering systems in a manner allowing relative axial translation therebetween.

### CROSS-REFERENCE TO RELATED APPLICATIONS

This application claims the benefit of priority under 35 U.S.C. § 119 to U.S. Provisional Application No. 63/354,015, filed Jun. 21, 2022, the entire disclosure of which is hereby incorporated by reference herein for all purposes.

### FIELD

The present disclosure relates generally to the field of medical devices and systems. More particularly, the present disclosure relates to devices, systems, and method for steering elongate members such as catheters.

### BACKGROUND

Devices, systems, and methods for delivering and/or deploying medical devices with minimally invasive techniques, such as percutaneously and/or transluminally, are desirable for avoiding more complex and invasive open surgical procedures. Various techniques which do not require open surgery utilize systems and devices with various flexible elongate members capable of navigating to an anatomical site within the body from a small insertion opening in a patient's body, transluminally through the body (such as through the vascular system), and to an anatomical site. A wide variety of intracorporeal medical devices have been developed for transluminal medical use, for example, intravascular use. Some of these systems and devices include guidewires, catheters, medical device delivery systems (e.g., for implantable devices such as tissue anchors, stents, grafts, replacement valves, etc.), and the like. Such systems may be multi-catheter/stacked catheter assemblies which include a plurality of tubular elongate members stacked one within the other (coaxially and/or coextensively within another flexible tubular elongate member). To reach an anatomical site within the body, various of the elongate members must be steerable to navigate through tortuous pathways within the body leading to the anatomical site. Improvements to handles for steering elongate members would be welcome in the art.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, a medical device delivery system includes a steerable flexible elongate member; and a two-way steering system operatively associated with the steerable flexible elongate member. In accordance with various further principles of the present disclosure, the two-way steering system includes a first flexible elongate steering element; a second flexible elongate steering element; and a control handle operatively associated with the first flexible elongate steering element and the second flexible elongate steering element to control steering of the steerable flexible elongate member via the first flexible elongate steering element and the second flexible elongate steering element.

In some embodiments, the control handle includes a control knob operatively associated with the first flexible elongate steering element and the second flexible elongate steering element to control movement of the steerable flexible elongate member. In some embodiments, the control knob is rotatable to control a steering gear system operatively coupled with the first flexible elongate steering element and the second flexible elongate steering element to control movement of the steerable flexible elongate member. In some embodiments, the steering gear system comprises a control knob pinion assembly operatively coupled with a steering gear pulley assembly; the control knob is operatively coupled with the control knob pinion assembly to control rotational movement of the control knob pinion assembly; and the steering gear pulley assembly is operatively coupled with the first flexible elongate steering element and the second flexible elongate steering element to control movement thereof to steer the steerable flexible elongate member. In some embodiments, the control knob has a directional control knob element controlling movement of the first flexible elongate steering element and the second flexible elongate steering element, and a friction control knob element controlling movement of the directional control knob element.

In some embodiments, the two-way steering system is a first two-way steering system operatively associated with the steerable flexible elongate member to steer the steerable flexible elongate member in a first steering plane; and the medical device delivery system further includes a second two-way steering system operatively associated with the steerable flexible elongate member to steer the steerable flexible elongate member in a second steering plane transverse to the first steering plane. In some embodiments, the medical device delivery system further includes a steerable flexible tubular elongate member; and a two-way steering system operatively associated with the steerable flexible tubular elongate member to steer the steerable flexible tubular elongate member in a steering plane; wherein the steerable flexible elongate member extends through the steerable flexible tubular elongate member. In some embodiments, the steerable flexible elongate member is longitudinally translatable within the steerable flexible tubular elongate member. In some embodiments, the two-way steering system operatively associated with the steerable flexible tubular elongate member is rotatable relative to the steering system operatively associated with the steerable flexible elongate member to adjust the orientation of the steering plane in which the steerable flexible tubular elongate member is steered. In some embodiments, the medical device delivery system further includes a first control knob operatively associated the first two-way steering system via a first steering gear system, and a second control knob operatively associated the second two-way steering system via a second steering gear system, wherein gears of the first steering gear system rotate about respective first gear system axes parallel to one another, and gears of the second steering gear system rotate about respective second gear system axes parallel to one another and transverse to the first gear system axes.

In accordance with various principles of the present disclosure, a control handle is provided to control steering of a steerable flexible elongate member with a first flexible elongate steering element and a second flexible elongate steering element operatively associated with the steerable flexible elongate member. In accordance with various principles of the present disclosure, the control handle has a control handle housing; and a control knob mounted on the control handle housing and operatively associated with the first flexible elongate steering element to steer the steerable flexible elongate member in a first direction, and to the second flexible elongate steering element to steer the steerable flexible elongate member in a second direction different from the first direction.

In some embodiments, the control knob includes a directional control knob element controlling movement of the first flexible elongate steering element and the second flexible elongate steering element, and a friction control knob element controlling movement of the directional control knob element.

In some embodiments, the control handle further includes a gear system having a control knob pinion assembly operatively associated with the control knob, and a steering gear pulley assembly operatively associated with the control knob pinion assembly and with the first flexible elongate steering element and the second flexible elongate steering element, wherein rotation of the control knob operates the gear system to control steering of the steerable flexible elongate member. In some embodiments, the steering gear pulley assembly includes a first steering gear pulley assembly operatively associated with the first flexible elongate steering element, and a second steering gear pulley assembly operatively associated with the second flexible elongate steering element; and at least one of the steering gear pulley assemblies includes a gear with a gear barrel extending therefrom and configured for coupling a flexible elongate steering element thereto, the steering gear pulley assembly configured to maintain the position of the flexible elongate steering element on the gear barrel without being entangled with the gear.

In some embodiments, the control knob steers the steerable flexible elongate member in a first steering plane, the control handle further including a control knob mounted on the control handle housing and operatively associated with a third flexible elongate steering element and a fourth flexible elongate steering element to steer the steerable flexible elongate member in a second steering plane transverse to the first steering plane.

In accordance with various principles of the present disclosure, a method of steering a steerable flexible elongate member of a medical device delivery system includes rotating a first control knob in a first direction to take up a first flexible elongate steering element about a first steering gear pulley assembly and to steer the steerable flexible elongate member in a first direction; and rotating the first control knob in a second direction to take up a second flexible elongate steering element about a second steering gear pulley assembly and to steer the steerable flexible elongate member in a second direction different from the first direction.

In some embodiments, rotating the first control knob steers the steerable flexible elongate member in a first steering plane. In some embodiments, rotating a second control knob in a first direction to take up a third flexible elongate steering element about a third steering gear pulley assembly and to steer the steerable flexible elongate member in a third direction; and rotating the second control knob in a second direction to take up a fourth flexible elongate steering element about a fourth steering gear pulley assembly and to steer the steerable flexible elongate member in a fourth direction different from the third direction. In some embodiments, rotating the first control knob steers the steerable flexible elongate member in a first steering plane and rotating the second control knob steers the steerable flexible elongate member in a second steering plane transverse to the first steering plane.

In some embodiments, the method further includes adjusting friction on the control knob with respect to a control handle to maintain the steerable flexible elongate member in a desired position.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a steering system and device in accordance with aspects of the present disclosure.

FIG. 2 illustrates a perspective view of a two-way steering system and device in accordance with aspects of the present disclosure.

FIG. 3 illustrates a two-way steering system and device as in FIG. 2 with portions thereof in phantom.

FIG. 4 illustrates an exploded view of a two-way steering system and device as in FIG. 3 with portions thereof in phantom.

FIG. 5 illustrates a perspective view of an example of an embodiment of a four-way steering system and device in accordance with aspects of the present disclosure.

FIG. 6 illustrates a four-way steering system and device as in FIG. 5 with portions thereof in phantom.

FIG. 7 illustrates a four-way steering system and device as in FIG. 5 with portions thereof in phantom.

FIG. 8 illustrates a two-way steering system as in FIG. 2 on an example of an embodiment of a stand.

FIG. 9 illustrates a four-way steering system as in FIG. 5 on an example of an embodiment of a stand.

FIG. 10 illustrates a schematic representation of an environment in which steerable flexible elongate members, illustrated in perspective, may be steered by a steering system formed in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “channel” or “bore” is not limited to a circular cross-section. As used herein, a “free end” of an element is a terminal end at which such element does not extend beyond.

Accessing regions within a body without cutting open the body (i.e., accessing such regions transluminally) often requires steering an elongate member into a complex position. For example, a procedure may involve navigating the elongate member in multiple directions, such as within a first plane and then within a second plane transverse to the first plane, and even within additional third, fourth, fifth, etc., planes. Various procedures such as, and without limitation, mitral valve therapies, utilize systems with more than one steerable elongate member, such as telescoped and/or stacked one within the other (coaxially and/or side-by-side and optionally within another tubular elongate member). The elongate members of such systems may need to be steered independently within different planes transverse to one another. For instance, an outer steerable tubular elongate member (such as a delivery catheter) may be steered in one or more planes to position a distal end of the steerable system at an anatomical site at which a procedure is to be performed with the use of systems and/or devices delivered by such steerable system. A distal portion of the outer steerable tubular elongate member may be steered and/or bent in a delivery plane for the system to place another system or device in a desired position with respect to the anatomical site (for instance, to center the distal end of the system on the mitral valve for repairing the mitral valve with another system or device). The outer steerable tubular elongate member generally remains bent in such delivery position (in a delivery plane) to maintain the distal end thereof in position for initial delivery of other systems or devices. The other systems or devices may be delivered and/or deployed by an inner steerable elongate member extending within the outer steerable tubular elongate member. The inner steerable elongate member is telescoped out of the outer steerable tubular elongate member, and then steered into another position. For instance, the inner steerable elongate member may be bent in a plane transverse to the delivery plane of the outer steerable tubular elongate member. The inner steerable elongate member may rotate and axially translate relative to the outer steerable tubular elongate member.

In accordance with various principles of the present disclosure, a system for delivering a medical device (referenced herein as a delivery system for the sake of convenience and without intent to limit) includes one or more steerable flexible elongate members and at least one steering system configured to maneuver an associated steerable flexible elongate member in at least one plane. It will be appreciated that terms such as maneuver (and other grammatical forms thereof) may be used interchangeably herein with such terms (and other grammatical forms thereof) as actuate, advance, articulate, bend, control, drive, flex, manipulate, move, navigate, operate, pull, retract, rotate, shift, steer, transition, translate, turn, etc., without intent to limit. The steering system includes one or more flexible elongate steering elements extending along a steering plane and operatively associated with an associated steerable flexible elongate member so that movement of the flexible elongate steering element causes movement of the steerable flexible elongate member at least within the steering plane. The steering system further includes a control handle operatively associated with the one or more flexible elongate steering elements. The control handle includes at least one control knob operatively coupled with the one or more flexible elongate steering elements to cause movement of the one or more flexible elongate steering elements. Such movement of the one or more flexible elongate steering elements may be within a steering plane, and optionally a common steering plane. For instance, the one or more flexible elongate steering elements may cause movement of the steerable flexible elongate member within the common steering plane in one or more directions (e.g., left and right in a horizontally-oriented plane, or up and down in a vertically-oriented plane). As such, the steering system may be considered a two-way steering system, and the steerable flexible elongate member may be considered a two-way steerable flexible elongate member. The steering plane may be considered to lie in a direction with respect to the anatomical site to which the delivery system is navigated. In some embodiments, the direction in which the common steering plane lies may be adjusted by rotating the steerable flexible elongate member, such as by rotating the delivery system.

In accordance with various principles of the present disclosure, a single control knob may control movement of one or more flexible elongate steering elements in a steering plane, such as a common steering plane. For instance, in some embodiments, rotation of a control knob in a first direction moves at least one flexible elongate steering element to steer an associated steerable flexible elongate member in a first steering direction. The first steering direction may be in generally the same direction as the first direction in which the control knob is moved, although the system need not be so limited. In some embodiments, rotation of the control knob in a first direction pulls on a first flexible elongate steering element and pays out a second flexible elongate steering element to steer the associated steerable flexible elongate member in a first steering direction. In some embodiments, rotation of the control knob in a second direction moves at least one flexible elongate steering element to steer an associated steerable flexible elongate member in a second steering direction. The second steering direction may be in a direction opposite to and either coplanar or noncoplanar with the first steering direction. The second steering direction may be in generally the same direction as the second direction in which the control knob is moved, although the system need not be so limited. In some embodiments, rotation of the control knob in the second direction pulls on a second flexible elongate steering element and pays out a first flexible elongate steering element to steer the associated steerable flexible elongate member in a second steering direction. The first steering direction and the second steering direction optionally are within a common steering plane.

In some aspects, the control handle includes a gear system configured to transfer rotation of the control knob to rotation of elements operatively coupled with the one or more flexible elongate steering elements. Operation of the control knob thus controls movement of the flexible elongate steering elements and thus movement of the steerable flexible elongate member with which the control handle and one or more flexible elongate steering elements are operatively associated. For example, the control knob may be operatively coupled with a pinion, and the one or more flexible elongate steering elements may be operatively coupled with a respective steering gear pulley engaging the control knob pinion. For example, the flexible elongate steering elements may wrap around a gear barrel operatively coupled with a respective steering gear pulley. The gear barrel may be a barrel, shaft, shank, etc., (such terms being used interchangeably herein without intent to limit) extending from a gear of the steering gear pulley along the rotation axis of the steering gear pulley. Rotation of the pinion gear may rotate a steering gear pulley with respect to which one or more flexible elongate steering elements are wound. As such, rotation of the control knob causes a flexible elongate steering element to be taken up/wound about a gear barrel or paid out from a gear barrel, such as depending on the direction in which the associated steering gear pulley is rotated, which is determined by the direction in which the control knob pinion is rotated. The gear system optionally includes one or more features configured to maintain the desired alignment of the flexible elongate steering element, such as with respect to the associated steerable flexible elongate member and/or steering plane and/or the steering gear pulley.

In some aspects, the control knob includes a directional control element and a friction control element. Movement of the directional control element controls movement of the associated one or more flexible elongate steering elements. Movement of the friction control element adjusts the movability of the directional control element, such as by increasing friction on and thereby reducing movability of the directional control element to maintain a selected steering position of the flexible elongate steering elements associated with the control knob.

Optionally, the delivery system includes a stand supporting one or more control handles of the delivery system. The delivery system stand may be configured to facilitate rotational and/or translational movement of one or more components of the delivery system. More particularly, the delivery system stand may be configured to support the delivery system as the delivery system is axially adjust along a longitudinal axis thereof, such as by adjusting components of the delivery system relative to one another. Additionally or alternatively, the delivery system stand may be configured to support the delivery system as the delivery system is rotated about its longitudinal axis.

In some embodiments, the two-way steerable flexible elongate member is tubular, and the delivery system further includes a four-way steerable flexible elongate member extending within the two-way steerable flexible tubular elongate member. Looked at another way, a two-way steerable flexible elongate member may have an additional set of steering elements (i.e., a second two-way steering system) to be a four-way steerable flexible elongate member. In some embodiments, such four-way steerable flexible elongate member may be positioned within a two-way steerable flexible tubular elongate member.

In accordance with various principles of the present disclosure, a four-way steerable flexible elongate member has a steering system similar to a steering system of a two-way steerable flexible elongate member. More particularly, a four-way steerable flexible elongate member may have a first steering system for steering the steerable flexible elongate member in a first steering plane, and a second steering system for steering the steerable flexible elongate member in a second steering plane. In some embodiments, the first steering system includes a first control knob operatively coupled to cause movement of the first of one or more flexible elongate steering elements. Such movement of the one or more flexible elongate steering elements may be within a steering plane, and optionally a common first steering plane. Similarly, the second steering system includes a second control knob operatively coupled to cause movement of the second of one or more flexible elongate steering elements. Such movement of the one or more flexible elongate steering elements may be within a steering plane, and optionally a common second steering plane. The first steering plane and the second steering plane may be transverse to, such as perpendicular to, each other. The first steering system and the second steering system may be housed in a common four-way steering system housing and thus may be considered components of an integrated four-way steering system. The components of each steering system may be substantially similar to the two-way steering system described above, reference being made thereto for the sake of brevity and simplicity.

A delivery system stand such as described above may be configured to accommodate a two-way steerable flexible tubular elongate member as well as a four-way steerable flexible elongate member slidable within the two-way steerable flexible tubular elongate member. The delivery system stand may be configured to support the delivery system during and after adjustment of the rotational position of one or more components of the delivery system and/or adjustment of the relative axial positions of components of the delivery system.

Various embodiments of a steering system and associated components and methods of use thereof will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

It will be appreciated that common features in the drawings are identified by common reference elements and, for the sake of brevity and convenience, and without intent to limit, the descriptions of the common features are generally not repeated. For purposes of clarity, not all components having the same reference number are numbered. Moreover, certain features in one embodiment may be used across different embodiments and are not necessarily individually labeled when appearing in different embodiments.

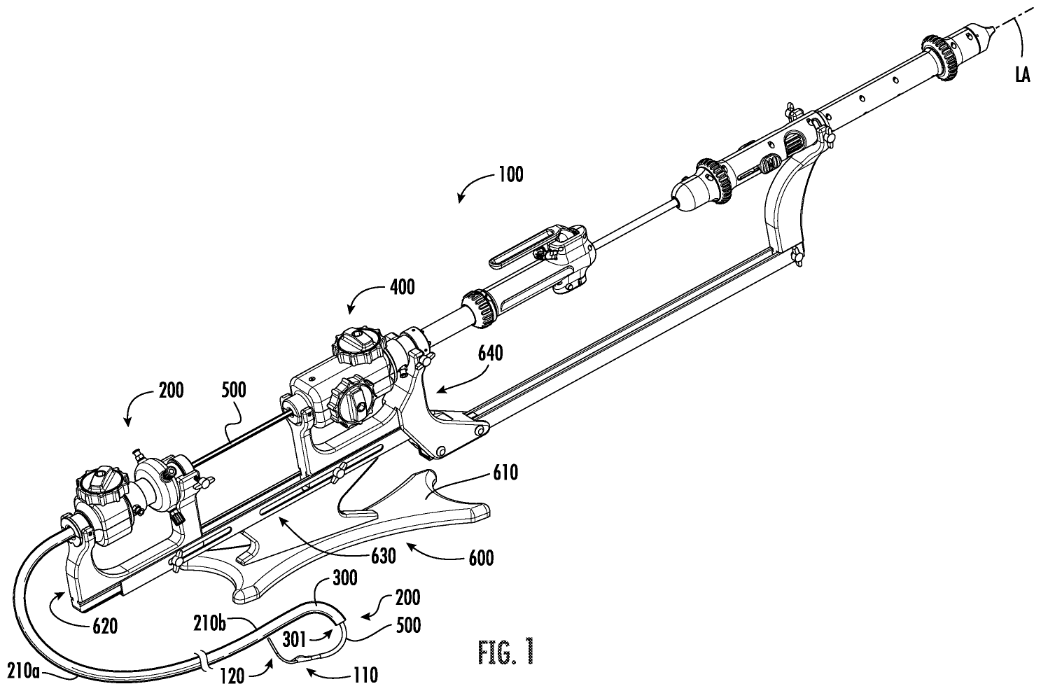

Turning now to the drawings, an example of an embodiment of a delivery system 100 for delivering medical devices and/or systems 110, 120 is illustrated in FIG. 1. The delivery system 100 includes at least one (a first) steering system, such as a two-way steering system 200, for a first steerable flexible elongate member 300. The delivery system 100 optionally includes an additional (a second) steering system, such as a four-way steering system 400, for an additional (a second) steerable flexible elongate member 500. As may be appreciated, in the illustrated example of an embodiment of a delivery system 100, the first steerable flexible elongate member 300 is tubular, with a lumen defined therethrough through which the second steerable flexible elongate member 500 extends. The first steerable flexible elongate member 300 is thus referenced herein as a steerable flexible tubular elongate member 300. In some embodiments, the second steerable flexible elongate member 500 is axially translatable through the steerable flexible tubular elongate member 300 to be extended therefrom or retracted therein, as discussed in further detail below.

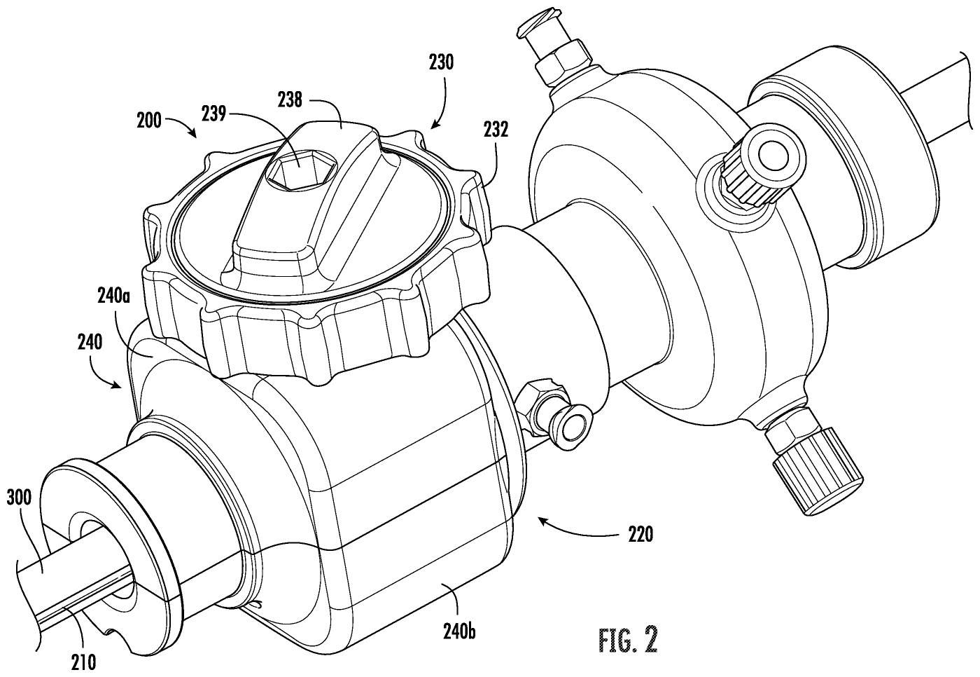

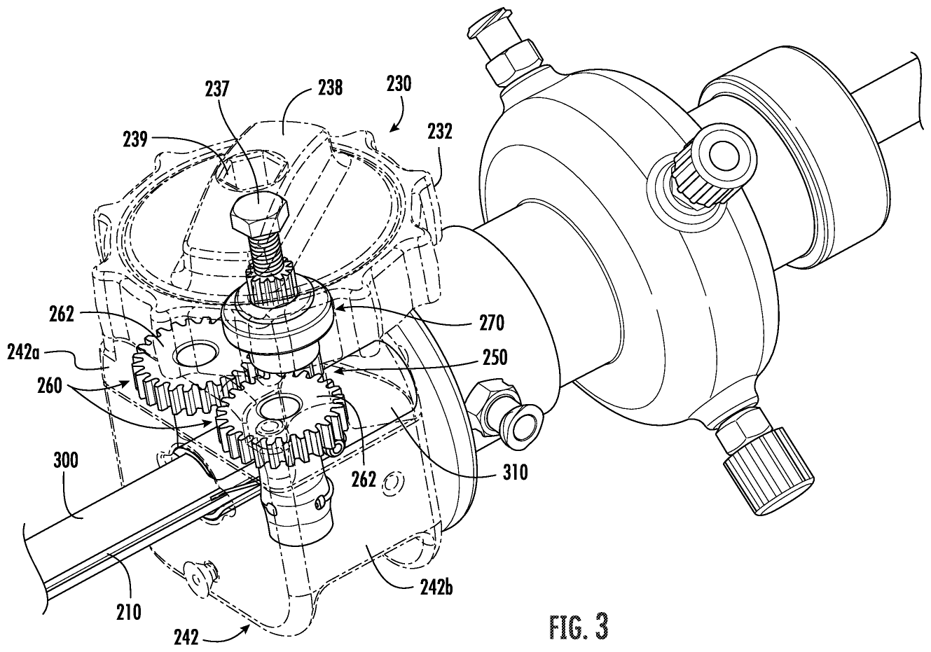

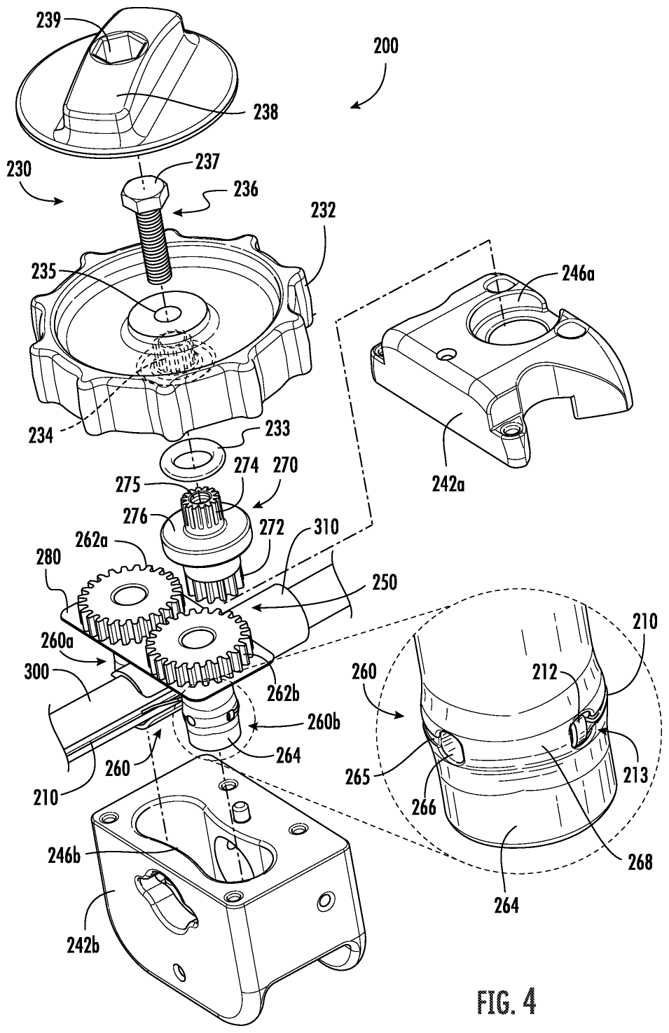

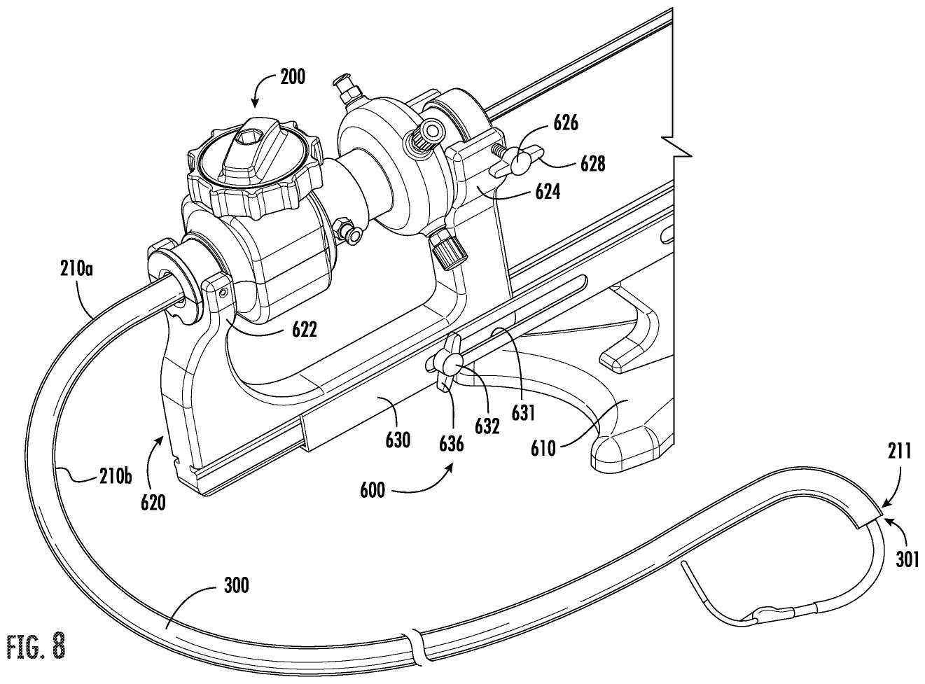

An example of an embodiment of a two-way steering system 200 formed in accordance with various principles of the present disclosure is illustrated in further detail in FIG. 2, FIG. 3, FIG. 4, and FIG. 8. The two-way steering system 200 includes one or more flexible elongate steering elements 210 operatively associated with the steerable flexible tubular elongate member 300, and a control handle 220. A flexible elongate steering element 210 is coupled to a steerable flexible tubular elongate member 300 (in any desired manner known to those of ordinary skill in the art) to control movement of the steerable flexible tubular elongate member 300. Optionally, the flexible elongate steering element 210 extends alongside the steerable flexible tubular elongate member 300. In an example of an embodiment illustrated in FIG. 8, a distal end 211 of a flexible elongate steering element 210 is coupled to a distal end 301 of the steerable flexible tubular elongate member 300 such that pulling of a proximal end 213 of the flexible elongate steering element 210 (see, e.g., detail view of FIG. 4) controls movement (e.g., steers) the steerable flexible tubular elongate member 300. The one or more flexible elongate steering elements 210 may be pull wires, ribbon cables, Bowden cables, etc., such as known by those of ordinary skill in the art for controlling or steering movement of a flexible elongate member. For instance, the one or more flexible elongate steering elements 210 may have a pull wire axially movable within an outer sheath (such as a Bowden cable), with the outer sheath coupled with (e.g., bonded to) the steerable flexible tubular elongate member 210 in any desired manner known to those of ordinary skill in the art.

The example of an embodiment of a control handle 220 illustrated in FIG. 2, FIG. 3, and FIG. 4 includes a control knob 230 mounted on a housing block 240. The housing block 240 may be formed of an upper housing cover 240a and a lower housing cover 240b coupled together (e.g., with fasteners such as screws) and forming an interior within which further working components of the two-way steering system 200 may be housed and supported. The housing covers 240a, 240b may be referenced alternately herein as carriage or carrier housings without intent to limit. In some embodiments, a further carrier bearing 242 is provided in the housing block 240. In some embodiments, the carrier bearing 242 is formed of an upper base component 242a and a lower base component 242b (coupled together with fasteners such as screws), such as illustrated in FIG. 3 and FIG. 4. The example of an embodiment of a carrier bearing 242 illustrated in FIG. 3 and FIG. 4 is configured to support the steerable flexible tubular elongate member 300. In some embodiments, the carrier bearing 242 holds the steerable flexible tubular elongate member 300 in place with one or more set screws (any set screw as known to those of ordinary skill in the art, and thus not illustrated in order to simplify the illustration of the other components of the two-way steering system 200). In some embodiments, a hypotube 310 is provided over and optionally bonded to the steerable flexible tubular elongate member 300 to provide a shaft mount which may protect the steerable flexible tubular elongate member 300. Additionally or alternatively, the carrier bearing 242 is configured to support and/or manage the one or more flexible elongate steering elements 210 (e.g., to prevent excess lengths of the one or more flexible elongate steering elements 210 spooling/extending out too far from the control handle 220). If a carrier bearing 242 is not provided, then the housing block 240 may be configured with various of the features of the carrier bearing 242.

In the example of an embodiment of a control handle 220 illustrated in FIG. 3 and FIG. 4, the housing block 240 is also configured to house a gear system 250 operatively associated with (e.g., controlled by) the control knob 230. The gear system 250 is also operatively associated with the one or more flexible elongate steering elements 210. The control knob 230 is thereby operatively associated with the one or more flexible elongate steering elements 210 so that operation of the control knob 230 (e.g., movement, such as rotation) actuates (e.g., operates, moves, steers, etc.) the one or more flexible elongate steering elements 210 to steer the steerable flexible tubular elongate member 300. The example of an embodiment of a gear system 250 illustrated in FIG. 3 and FIG. 4 includes one or more steering gear pulley assemblies 260a, 260b and a control knob pinion assembly 270. Each steering gear pulley assembly 260 is operatively associated with an associated flexible elongate steering element 310. In the illustrated example of an embodiment, the steering gear pulley assembly 260 includes a steering gear 262 with a pulley shaft extending therefrom and referenced herein as a gear barrel 264. The flexible elongate steering element 210 may be coupled with the gear barrel 264 in any of a variety of manners known to those of ordinary skill in the art. For instance, in the example of an embodiment illustrated in the detail view of FIG. 4, the flexible elongate steering element 210 may be insertable through a slot 265 formed in the gear barrel 264. The flexible elongate steering element 210 may have an enlarged proximal end 213, such as by being provided or formed with a crimp or ferrule 212, as also illustrated in the detail view of FIG. 4. Such enlarged proximal end 213 (e.g., the crimp or ferrule 212) may be fitted or seated within a pocket 266 in the gear barrel 264. The enlarged proximal end 213 may be further secured within the pocket 266, such as by adhering, welding, brazing, soldering, etc., or any other manner known by those of ordinary skill in the art. In some embodiments, the gear barrel 264 includes a bulge or bump 268 positioned to maintain the position of the flexible elongate steering element 210 with respect to the gear barrel 264. In some embodiments, a shim 280 is provided between the flexible elongate steering element 210 and at least the gear 262 of the steering gear pulley assembly 260 with which the flexible elongate steering element 210 is associated. The shim 280 is positioned to prevent shifting or creeping of the flexible elongate steering element 210 into and entangling with the gear 262.

In accordance with various principles of the present disclosure, the steering gear pulley assembly 260 is configured such rotation thereof causes movement of an associated flexible elongate steering element 210 to steer an associated steerable flexible tubular elongate member 300. More particularly, in the illustrated example of an embodiment, the flexible elongate steering element 210 is coupled with the gear barrel 264 of the steering gear pulley assembly 260 so that rotation of the steering gear 262 of the steering gear pulley assembly 260 causes rotation of the gear barrel 264 to cause the flexible elongate steering element 210 to be spooled/pulled onto or paid out from the gear barrel 264, depending on the direction of rotation of the steering gear pulley assembly 260. For instance, pulling on a flexible elongate steering element 210 extending along a first side of a steerable flexible tubular elongate member 300 steers the steerable flexible tubular elongate member 300 in a first direction towards the first side (such as by causing the steerable flexible tubular elongate member 300 to bend or flex towards the first side). Likewise, pulling on a flexible elongate steering element 210 extending along a second side of a steerable flexible tubular elongate member 300 steers the steerable flexible tubular elongate member 300 in a second direction towards the second side (such as by causing the steerable flexible tubular elongate member 300 to bend or flex towards the second side).

In accordance with various principles of the present disclosure, the control knob 230 is operatively associated with the gear system 250 to operate or control the gear system 250 to steer the steerable flexible tubular elongate member 300 via one or more flexible elongate steering elements 210. In the example of an embodiment illustrated in FIG. 3, the control knob 230 is operatively associated with a control knob pinion assembly 270, such as to control rotational movement of the control knob pinion assembly 270. As may be appreciated with reference to FIG. 4, the control knob pinion assembly 270 includes a pinion 272 engaged with the steering gear 262 of the steering gear pulley assembly 260. In the example of an embodiment of a two-way steering system 200 illustrated in FIG. 4, the pinion 272 is operatively engaged (e.g., meshes) with a steering gear 262a of a first steering gear pulley assembly 260a as well as with a steering gear 262b of a second steering gear pulley assembly 260b. As such, rotation of the pinion 272 causes rotation of both steering gear pulley assemblies 260a, 260b of the gear system 250. As may be also appreciated with reference to FIG. 3 and FIG. 4, the control knob 230 is coupled with the control knob pinion assembly 270 to rotate the pinion 272. More particularly, the control knob 230 includes a directional control knob element 232 with splines 234 engaging corresponding splines 274 on the control knob pinion assembly 270 to impart rotational movement of the control knob 230 to the control knob pinion assembly 270. The directional control knob element 232 may be knurled or otherwise to facilitate manual rotation thereof. The directional control knob element 232 is coupled with the control knob pinion assembly 270 via a bolt 236 extending through a bolt hole 235 through the directional control knob element 232 and through a bolt hole 275 through the control knob pinion assembly 270. The head 237 of the bolt 236 may rest above the directional control knob element 232 and be larger than the bolt hole 235 to couple the directional control knob element 232 to the control knob pinion assembly 270.

In accordance with various principles of the present disclosure, it may be desirable to hold the steerable flexible tubular elongate member 300 in a desired orientation or configuration. For instance, the steerable flexible tubular elongate member 300 may be an introducer or delivery sheath delivering further components or systems of the delivery system 100 to an anatomical site. Once the steerable flexible tubular elongate member 300 has reached the general vicinity of the anatomical site, it may be desirable to fix or hold the steerable flexible tubular elongate member 300 in a particular configuration so that further devices or systems (e.g., a four-way steerable flexible elongate member 500 controlled by a four-way steering system 400 noted above, and/or other devices or systems, such as medical devices and/or systems 110, 120 described in further detail below). In accordance with various principles of the present disclosure, the control knob 230 may include, in addition to a directional control knob element 232, a friction control knob element 238. The friction control knob element 238 is configured to limit, restrict, lock, inhibit, etc. (such terms and other grammatical forms thereof being used interchangeably herein without intent to limit) movement of the directional control knob element 232 to likewise limit, restrict, lock, inhibit, etc., movement of the steering gear pulley assembly 260 and the one or more flexible elongate steering elements 210. In the example of an embodiment illustrated in FIG. 3 and FIG. 4, the friction control knob element 238 is provided on (e.g., rests on) the directional control knob element 232 and is movable towards or away from the directional control knob element 232 to move the directional control knob element 232 towards or away from the upper base component 242a of the carrier bearing 242. In some embodiments, the bolt 236 is a threaded bolt threadedly engaged with the directional control knob element 232, and a hex head 237 of the bolt 236 fits within a non-circular (e.g., complementary hex-shaped) hole 239 within the friction control knob element 238. Rotation of the friction control knob element 238 thus rotates the bolt 236 to cause the directional control knob element 232 to move closer to or further from the carrier bearing 242. In some embodiments, a frictional element 233 (e.g., an o-ring) is provided between the directional control knob element 232 and the upper base component 242a. Engagement of the directional control knob element 232 with the frictional element 233 as the friction control knob element 238 is rotated increases friction on the directional control knob element 232 to hold the directional control knob element 232 in a desired angular (rotational) position. In some embodiments, the frictional element 233 rests on a shoulder 276 radially extending from the control knob pinion assembly 270. In some embodiments, the shoulder 276 of the control knob pinion assembly 270 is seated within a seat 246a formed in the upper base component 242a of the carrier bearing 242 (towards which the directional control knob element 232 is advanced). The seating of the shoulder 276 within the upper base component 242a may serve to stabilize the control knob pinion assembly 270 with respect to the carrier bearing 242 and/or the housing block 240 of the control handle 220. As the friction control knob element 238 moves the directional control knob element 232 towards the upper base component 242a, the frictional element 233 is compressed to increase friction with respect to the directional control knob element 232 and thereby to inhibit movement of the directional control knob element 232 and consequently the gear system 250 and steering gear pulley assembly 260 and one or more flexible elongate steering elements 210 and steerable flexible tubular elongate member 300.

It will be appreciated that the upper base component 242a and the lower base component 242b of the carrier bearing 242 may include various additional features for supporting the other components of the two-way steering system 200. For instance, the lower base component 242b may include one or more seats 246b in which the steering gears 262 of the steering gear pulley assembly 260 may be supported (e.g., to stabilize the steering gear pulley assemblies 260).

In use, an example of an embodiment of a two-way steering system 200 such as illustrated in FIG. 1, FIG. 2, FIG. 3, FIG. 4, and FIG. 8 has a first flexible elongate steering element 210a extending along a first side of a steerable flexible tubular elongate member 300 and a second flexible elongate steering element 210b extending along a second side of the steerable flexible tubular elongate member 300 (see, e.g., FIG. 1 and FIG. 8). The flexible elongate steering elements 210a, 210b are operatively coupled with respective first and second steering gear pulley assemblies 260a, 260b. The steering gear pulley assemblies 260a, 260b are operatively coupled with the control knob 230 (such as via the control knob pinion assembly 270) such that rotation of the control knob 230 controls steering of the steerable flexible tubular elongate member 300 as desired. In some embodiments, rotation of the control knob 230 in a first direction (e.g., clockwise rotation) pulls the first flexible elongate steering element 210a to steer the steerable flexible tubular elongate member 300 in the first direction (e.g., to the right), and rotation of the control knob 230 in a second direction (e.g., counterclockwise rotation) pulls the second flexible elongate steering element 210b to steer the second flexible elongate steering element 210b in the second direction (e.g., to the left). However, the present disclosure need not be so limited and other configurations are within the scope and spirit of the present disclosure. The one or more flexible elongate steering elements 210 may be considered to lie in a steering plane and to control movement of the steerable flexible tubular elongate member 300 within such steering plane.

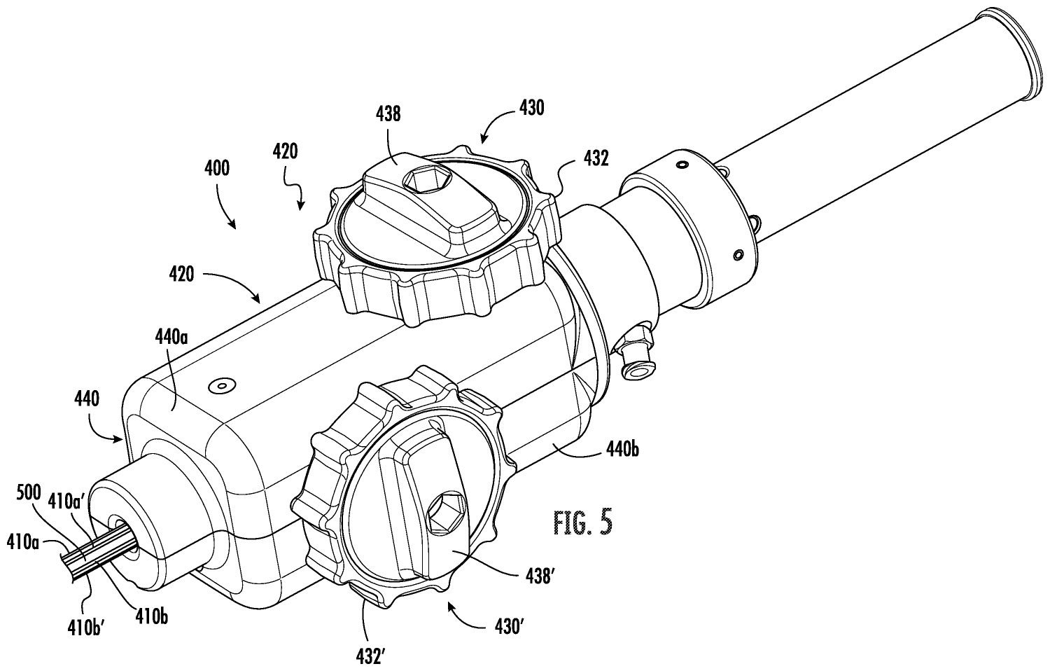

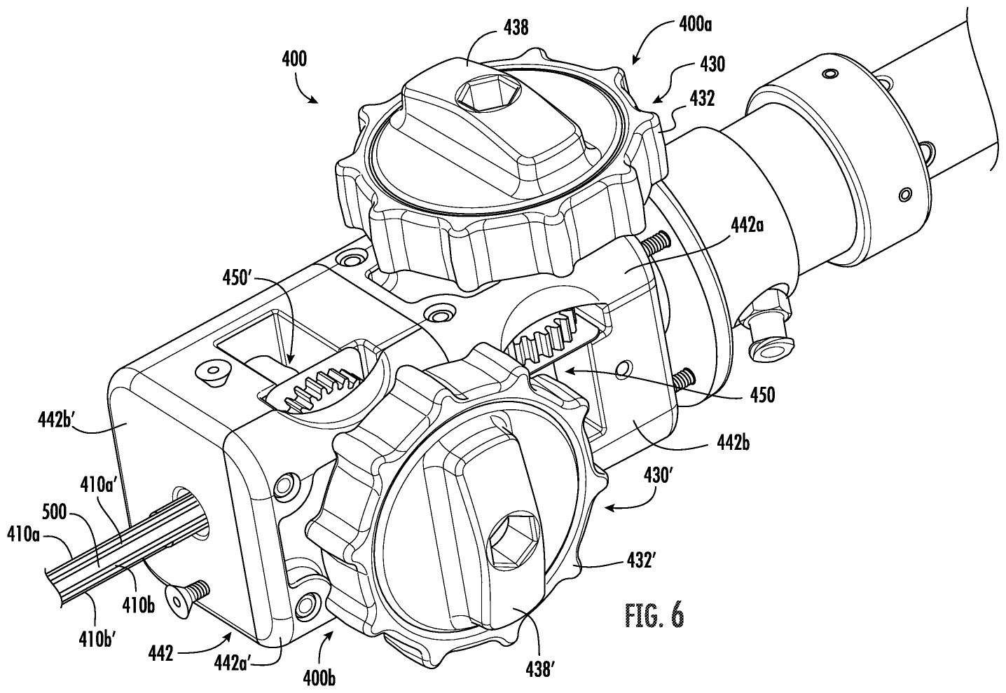

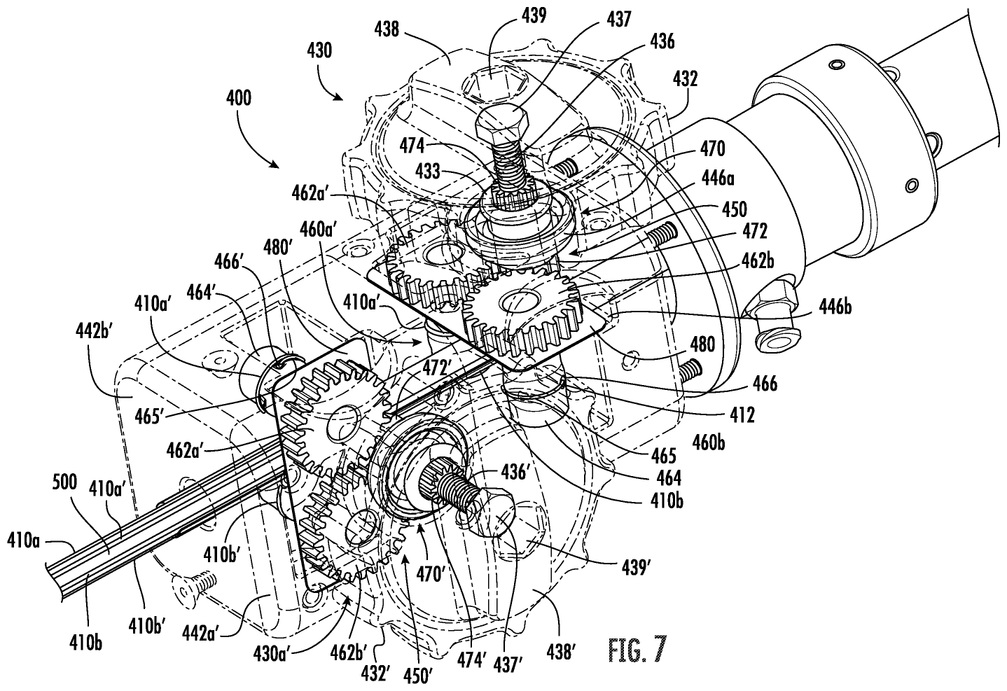

In accordance with various principles of the present disclosure, a four-way steering system 400 such as illustrated generally in FIG. 1 may be formed in a similar manner as the two-way steering system 200 described above. More particularly, the four-way steering system 400 illustrated in further detail in FIG. 5, FIG. 6, and FIG. 7, is formed of two sets of steering systems each similar to the two-way steering system 200 of FIGS. 2-4 and oriented with their respective steering planes transverse to each other, such as perpendicular to each other. The first section 400a of the four-way steering system 400 has components substantially the same as components of the two-way steering system 200 described above. Accordingly, similar components are indicated by similar reference numerals increased by 200. The second section 400b of the four-way steering system 400 has components substantially the same as components of the two-way steering system 200 described above and the first section 400a of the four-way steering system 400. Accordingly, components of the second section 400b of the four-way steering system 400 are indicated by similar reference numerals as used with reference to the first section 400a of the four-way steering system 400 and with a prime (′) appended thereto. For the sake of brevity, reference is made to the above descriptions of such elements without intent to limit. As may be appreciated, the four-way steering system 400 defines a first steering plane along which the first section 400a of the four-way steering system 400 may steer the steerable flexible elongate member 500 with the assistance of one or more flexible elongate steering elements 410a and 410b, and a second steering plane along which the second section 400b of the four-way steering system 400 may steer the steerable flexible elongate member 500 with the assistance of one or more flexible elongate steering elements 410a′ and 410b′ in a manner similar to that described above with reference to the two-way steering system 200. The first steering plane and the second steering plane are transverse to each other, such as perpendicular to each other. The control knob pinion assembly 470 and steering gear pulley assembly 460 of the gear system 450 of the first section 400a of the four-way steering system 400 rotate about first rotational axes parallel to one another. The control knob pinion assembly 470′ and steering gear pulley assembly 460′ of the gear system 450′ of the second section 400b of the four-way steering system 400 rotate about second rotational axes parallel to one another and transverse to (e.g., perpendicular to) the first rotational axes.

To facilitate positioning and/or movement of the delivery system 100, a stand may be provided to support at least a two-way steering system 200 of the delivery system 100, as illustrated in FIG. 1. The illustrated example of an embodiment of a stand 600 includes a base 610 supporting a first steering system support 620 configured to support the two-way steering system 200. Optionally, the stand 600 facilitates moving of the two-way steering system 200, such as during operation of the two-way steering system 200. For instance, the two-way steering system 200 may be rotated during operation thereof to adjust the orientation of the steering plane of the two-way steering system 200, such as the orientation of the steering plane in which the steerable flexible tubular elongate member 300 is navigated with respect to an anatomical site. In the example of an embodiment of a first steering system support 320 illustrated in FIG. 1, and in further detail in FIG. 8, a first yoke 622 and a second yoke 624 are configured and positioned to support the two-way steering system 200. The two-way steering system 200 may be rotated within the first steering system support 620 to adjust the steering plane in which the steerable flexible tubular elongate member 300 is steered. The yokes 622, 624 may thus be configured to allow rotation of the two-way steering system 200 while being supported by the yokes 322, 324. At least one locking element 626 may be provided to fix the two-way steering system 200 with respect to at least one of the yokes 622, 624 once positioned in the desired orientation. The locking element 626 may include a thumb nut 628 to facilitate tightening or loosening of the locking elements 626 to fix or release the angular position of the two-way steering system 200 with respect to the stand 600, such as manually (by hand and without the need for an additional tool). As such, angular position of the two-way steering system 200 may be readily adjusted during a procedure without interruptions caused by the need for separate additional adjustment tools.

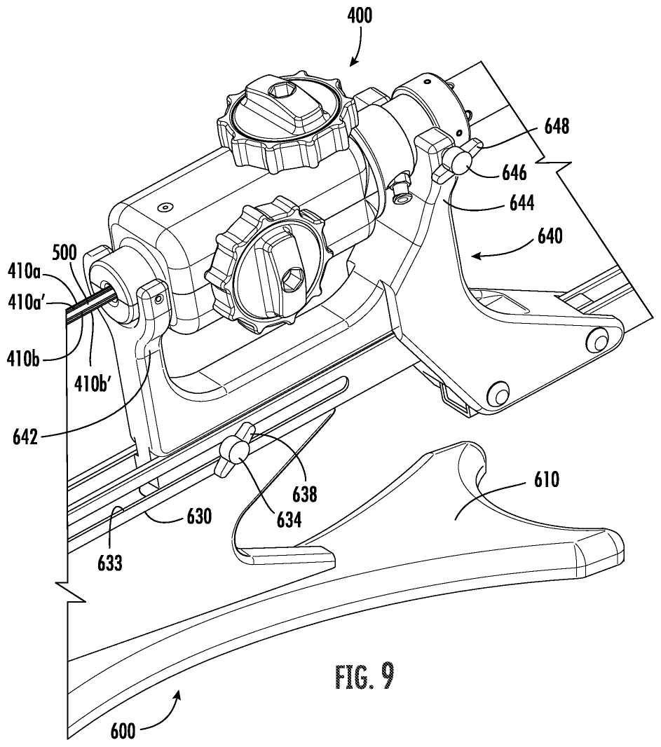

In delivery systems 100 including a four-way steering system 400 as well, the first steering system support 620 is coupled via a coupler 630 to a second steering system support 640. The example of an embodiment of a stand 600 illustrated in FIG. 1 includes a second steering system support 640 configured to support the four-way steering system 400. In the example of an embodiment of a second steering system support 640 illustrated in FIG. 1, and in further detail in FIG. 9, a first yoke 642 and a second yoke 644 are configured and positioned to support the four-way steering system 400. At least one locking element 646 may be provided to fix the four-way steering system 400 with respect to at least one of the yokes 642, 644 once positioned in the desired orientation or position within the second steering system support 640.

As discussed above, the four-way steering system 400 may be linearly or longitudinally translatable with respect to the two-way steering system 200, such as to extend or to retract the steerable flexible elongate member 500 with respect to the steerable flexible tubular elongate member 300. In some embodiments, the stand 600 is configured not only to support both the two-way steering system 200 and the four-way steering system 400, but also to facilitate relative movement therebetween (e.g., along the longitudinal axis LA of the delivery system 100). For instance, the first steering system support 620 and the second steering system support 640 may be coupled together via a coupler 630 which may be configured to allow a longitudinally slidable connection between the first steering system support 620 and the second steering system support 640. For instance, the first steering system support 620 may be slidably mounted with respect to the coupler 630 with a slidable locking element 632 slidable through a slot 631, such as illustrated in further detail in FIG. 8. Similarly, the second steering system support 640 may be slidably mounted with respect to the coupler 630 with a slidable locking element 634 slidable through a slot 633, such as illustrated in further detail in FIG. 9. The slidable locking elements 632, 634 may include respective thumb nuts 636, 638 to facilitate loosening or tightening of the slidable locking elements 632, 634 with respect to the respective slots 631, 633, such as manually (by hand and without the need for an additional tool). As such, the stand 600 facilitates ready adjustment of the delivery system 100 during a procedure without interruptions caused by the need for separate additional adjustment tools.

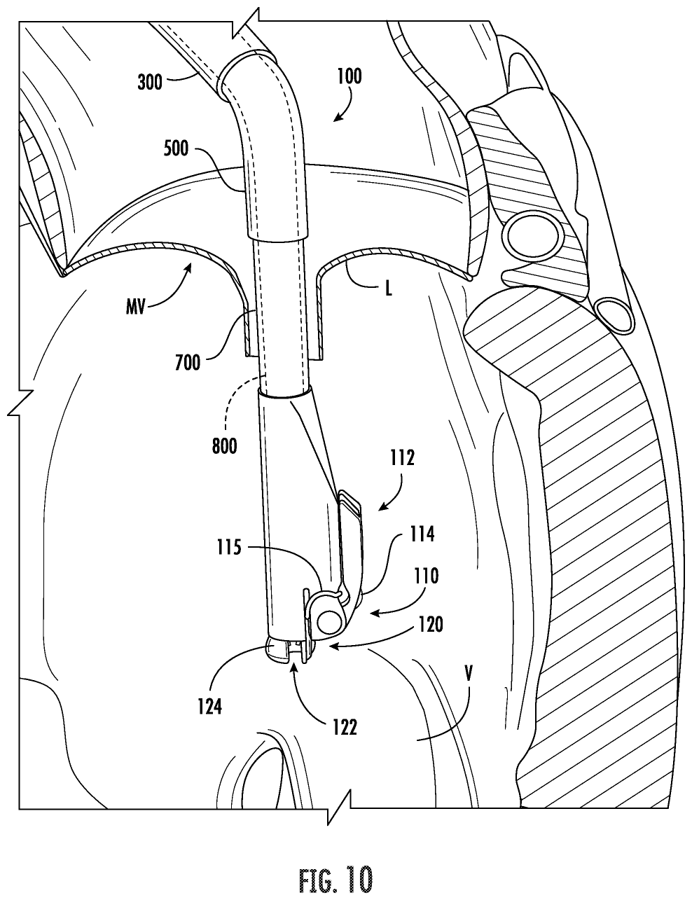

Principles of the present disclosure as described above are applicable in a wide range of delivery systems, such as devices delivered transluminally/transcatheterally. An example of a delivery system 100 configured to deliver and to deploy devices for cardiac procedures such as mitral valve and/or cardiac leaflet repair, and to which principles of the present disclosure may be applied, is illustrated in FIG. 10. In the illustrated example of an embodiment, a steerable flexible tubular elongate member 300 as described above may be used as an introducer sheath or guide sheath to deliver treatment devices and systems to a heart (such as a heart ventricle V). Furthermore, in the illustrated example of an embodiment, a steerable flexible elongate member 500 as described above may be used as a four-way steerable delivery catheter to deliver medical devices and/or systems 110, 120 to the heart ventricle V such as to repair a valve (e.g., the mitral valve MV) thereof. One or more device delivery catheters 700, 800 are extendable through the steerable flexible tubular elongate member 300 and the steerable flexible elongate member 500. The device delivery catheters 700, 800 generally are not steerable on their own such that their positions and configurations are determined by the steerable flexible tubular elongate member 300 and the steerable flexible elongate member 500. The illustrated example of an embodiment of a device delivery catheter 700 is configured to deliver a leaflet clip delivery and deployment system 110. The leaflet clip delivery and deployment system 110 includes a leaflet clip spreader 112 configured to deliver a leaflet clip 112 to a heart leaflet L. The illustrated example of an embodiment of a delivery device catheter 800 is configured to delivery an anchor delivery and deployment device 120 configured to deliver and to deploy a cardiac anchor 122 (optionally with the use of an anchor garage 124) with respect to cardiac tissue, such as the ventricle V. An artificial chordae tendineae 115 may be coupled to the leaflet clip 114 and thus to the leaflet L, and anchored to the ventricle V by the anchor 122.

It will be appreciated that principles of the present disclosure may be applied to a steerable flexible elongate member used in a multi-catheter stack-up assembly 100 such as illustrated in FIG. 1 or separately or with other devices, systems, instruments, etc. Although a steering system formed in accordance with various principles of the present disclosure is illustrated in an example environment of a heart, it will be appreciated that other environments are within the scope and spirit of the present disclosure. Principles of the present disclosure are particularly useful in devices and systems for accessing an anatomical site within a patient's body via a transluminal access within the body and without cutting open the body, although not necessarily so limited. Various further benefits of the various aspects, features, components, and structures of steerable devices, systems, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A medical device delivery system comprising:
a steerable flexible elongate member; and
a two-way steering system operatively associated with said steerable flexible elongate member and comprising:
a first flexible elongate steering element;
a second flexible elongate steering element; and
a control handle operatively associated with the first flexible elongate steering element and the second flexible elongate steering element to control steering of the steerable flexible elongate member via the first flexible elongate steering element and the second flexible elongate steering element.

2. The medical device delivery system of claim 1, wherein said control handle includes a control knob operatively associated with said first flexible elongate steering element and said second flexible elongate steering element to control movement of said steerable flexible elongate member.

3. The medical device delivery system of claim 2, wherein said control knob is rotatable to control a steering gear system operatively coupled with said first flexible elongate steering element and said second flexible elongate steering element to control movement of said steerable flexible elongate member.

4. The medical device delivery system of claim 3, wherein:
said steering gear system comprises a control knob pinion assembly operatively coupled with a steering gear pulley assembly;
said control knob is operatively coupled with said control knob pinion assembly to control rotational movement of said control knob pinion assembly; and
said steering gear pulley assembly is operatively coupled with said first flexible elongate steering element and said second flexible elongate steering element to control movement thereof to steer said steerable flexible elongate member.

5. The medical device delivery system of claim 2, wherein said control knob comprises a directional control knob element controlling movement of said first flexible elongate steering element and said second flexible elongate steering element, and a friction control knob element controlling movement of said directional control knob element.

6. The medical device delivery system of claim 1, wherein:
said two-way steering system is a first two-way steering system operatively associated with said steerable flexible elongate member to steer said steerable flexible elongate member in a first steering plane; and
said medical device delivery system further comprises a second two-way steering system operatively associated with said steerable flexible elongate member to steer said steerable flexible elongate member in a second steering plane transverse to said first steering plane.

7. The medical device delivery system of claim 6, further comprising:
a steerable flexible tubular elongate member; and
a two-way steering system operatively associated with said steerable flexible tubular elongate member to steer said steerable flexible tubular elongate member in a steering plane;
wherein said steerable flexible elongate member extends through said steerable flexible tubular elongate member.

8. The medical device delivery system of claim 7, wherein said steerable flexible elongate member is longitudinally translatable within said steerable flexible tubular elongate member.

9. The medical device delivery system of claim 7, wherein said two-way steering system operatively associated with said steerable flexible tubular elongate member is rotatable relative to said steering system operatively associated with said steerable flexible elongate member to adjust the orientation of the steering plane in which said steerable flexible tubular elongate member is steered.

10. The medical device delivery system of claim 6, further comprising a first control knob operatively associated said first two-way steering system via a first steering gear system, and a second control knob operatively associated said second two-way steering system via a second steering gear system, wherein gears of said first steering gear system rotate about respective first gear system axes parallel to one another, and gears of said second steering gear system rotate about respective second gear system axes parallel to one another and transverse to said first gear system axes.

11. A control handle for controlling steering of a steerable flexible elongate member with a first flexible elongate steering element and a second flexible elongate steering element operatively associated with said steerable flexible elongate member, said control handle comprising:
a control handle housing; and
a control knob mounted on said control handle housing and operatively associated with the first flexible elongate steering element to steer the steerable flexible elongate member in a first direction, and to the second flexible elongate steering element to steer the steerable flexible elongate member in a second direction different from the first direction.

12. The control handle of claim 11, wherein said control knob comprises a directional control knob element controlling movement of the first flexible elongate steering element and the second flexible elongate steering element, and a friction control knob element controlling movement of said directional control knob element.

13. The control handle of claim 11, further comprising a gear system comprising a control knob pinion assembly operatively associated with said control knob, and a steering gear pulley assembly operatively associated with the control knob pinion assembly and with the first flexible elongate steering element and the second flexible elongate steering element, wherein rotation of said control knob operates said gear system to control steering of the steerable flexible elongate member.

14. The control handle of claim 13, wherein:
said steering gear pulley assembly comprises a first steering gear pulley assembly operatively associated with the first flexible elongate steering element, and a second steering gear pulley assembly operatively associated with the second flexible elongate steering element; and
at least one of said steering gear pulley assemblies comprises a gear with a gear barrel extending therefrom and configured for coupling a flexible elongate steering element thereto, the steering gear pulley assembly configured to maintain the position of the flexible elongate steering element on the gear barrel without being entangled with the gear.

15. The control handle of claim 11, wherein said control knob steers the steerable flexible elongate member in a first steering plane, said control handle further comprising a control knob mounted on said control handle housing and operatively associated with a third flexible elongate steering element and a fourth flexible elongate steering element to steer the steerable flexible elongate member in a second steering plane transverse to the first steering plane.

16. A method of steering a steerable flexible elongate member of a medical device delivery system, said method comprising:
rotating a first control knob in a first direction to take up a first flexible elongate steering element about a first steering gear pulley assembly and to steer the steerable flexible elongate member in a first direction; and
rotating the first control knob in a second direction to take up a second flexible elongate steering element about a second steering gear pulley assembly and to steer the steerable flexible elongate member in a second direction different from the first direction.

17. The method of claim 16, wherein rotating the first control knob steers the steerable flexible elongate member in a first steering plane.

18. The method of claim 17, further comprising:
rotating a second control knob in a first direction to take up a third flexible elongate steering element about a third steering gear pulley assembly and to steer the steerable flexible elongate member in a third direction; and
rotating the second control knob in a second direction to take up a fourth flexible elongate steering element about a fourth steering gear pulley assembly and to steer the steerable flexible elongate member in a fourth direction different from the third direction.

19. The method of claim 18, wherein rotating the first control knob steers the steerable flexible elongate member in a first steering plane and rotating the second control knob steers the steerable flexible elongate member in a second steering plane transverse to the first steering plane.

20. The method of claim 16, further comprising adjusting friction on said control knob with respect to a control handle to maintain the steerable flexible elongate member in a desired position.
